(19) United States
(12) Patent Application Publication
(10) Pub. No.: US 2008/0032315 A1
(43) Pub. Date: Feb. 7, 2008

(54) Method and Kit for Determining the Immunisation Status of a Person

(75) Inventor: Claude Escarguel, Sanary Sur Mer (FR)

Correspondence Address: DENNISON, SCHULTZ & MACDONALD, 1727 KING STREET, SUITE 105, ALEXANDRIA, VA, 22314, US

(73) Assignee: INODIAG, La Ciotat (FR)

(21) Appl. No.: 11/572,642

(22) PCT Filed: Jul. 29, 2005
(86) PCT No.: PCT/FR05/01990

§ 371 Date: Jan. 25, 2007

(30) Foreign Application Priority Data
Aug. 6, 2004 (FR) 04 08600

(51) Int. Cl. G01N 33/566; G01N 33/543

(52) U.S. Cl. 435/7.21; 435/7.2; 436/518

(57) ABSTRACT

The present invention concerns a method for determining vaccine status by quantifying type-IgG serum antibodies of a plurality of pathogenic agents, characterized in that the steps are conducted of: 1—contacting one same said serum sample to be tested with one same solid substrate on which a plurality of said vaccine antigens is fixed corresponding to a plurality of vaccine antigens of different pathogenic agents, at different areas of the substrate, in the presence of at least one detection substance reacting by complexing with said IgG-type specific antibodies and not reacting with said vaccine antigens, and 2—the concentration of said IgG-type specific antibodies is determined.

The present invention concerns a diagnosis method and kit for the serological determination of a person's vaccine status. Vaccination consists of using a suitable administration route to administer one or more antigens of a bacterial, viral or parasitic pathogenic agent, optionally associated with one or more substances stimulating the immune response to this or these antigens (vaccine adjuvants).

Irrespective of the vaccination mode, the objective is to provide vaccinated persons with a specific immune response to the pathogen comprising memory immunity, i.e. able to respond within a few hours to any new contact with the pathogen, thereby efficiently protecting the person against the pathogen. One particular, essential condition for immunity imparted by vaccination is the production of antibodies specific to the vaccine antigen.

The protection afforded by vaccination is not permanent, and only remains acquired for a lapse of time varying from a few months to a few decades. The pathogen against which vaccination is made may show periodical changes in its antigenic composition, which fully or partly cancel the protection provided by vaccination with the previous antigenic composition. Also, the relative prevalence of different antigenic variants (serotypes) of the pathogen in relation to time and the country under consideration, requires modifications to be made to the composition of the vaccine in order to incorporate the antigens of newly prevalent serotypes. This is illustrated by the vaccine recommendations specifically drawn up for travellers to countries in which these pathogens are highly endemic [Mackell S M. Vaccinations for the pediatric traveller. Clin. Infect. Dis. 2003, 37:1508-1515; Kirkpatrick B. D. & Kemper Alston W. Current immunization for travel. Current Opinion in Infectious Diseases 2003, 16:369-374]. Also, the lifetime of the protective antibodies induced by the vaccine is limited to a few years in the absence of a new antigenic stimulation by contact with the pathogen, or via a new vaccination. Similarly, individual response may vary depending on the immunity status at the time of vaccination and according to route of administration. These reasons: antigenic changes of the pathogen, gradual disappearance of specific protective antibodies and response heterogeneity, require the re-vaccination of persons in the form of booster vaccines.

Vaccination and booster vaccination can be analyzed taking into account the known side effects of some vaccines, cost/benefit analysis, and a person's free choice for non-compulsory vaccines, in accordance with recommendations published in the form of a vaccine schedule specifying the conditions for vaccine administration and any necessary boosters [anonymous: Calendrier vaccinal 2003, Avis du Conseil Supérieur d'Hygiène Publique de France. Bulletin Epidémiologique hebdomadaire 2003, 6:33-36]. The policy of systematic re-vaccination of the population is increasingly placed in doubt, further to cost-benefit analyses and pressure by public opinion. A decision for re-vaccination that is individually adapted to the immunity status of a person is therefore desirable. The cost of vaccination is a major stake not only for industrialized countries, but more especially for developing countries [Carabin H & Edmunds W J. Future Savings from measles eradication in industrialized countries. J. Infect. Dis. 2003, 187 (suppl 1):529-535].

Determining the presence, even the level, of specific protective antibodies in the serum of a person to be vaccinated should be a key factor in deciding whether to give a vaccine or booster. If a person's serum contains antibodies specific to the pathogen at a protective concentration, there will be no benefit but rather a risk in giving this person a booster vaccine.

The search for the presence of specific IgG antibodies is currently being conducted in laboratories for the measles, mumps and rubella viruses. Laboratory determination of specific antibody concentration is only carried out for the hepatitis B virus: an anti-HBs antibody level of >10 mIU/ml is considered to be protective against infection with hepatitis B virus, and does not warrant a booster vaccine. For the detection of anti-tetanus antibodies (directed against the tetanus toxin) a few quick tests based on a passive hemagglutination technique (Vacci-Test® Pasteur®) or on immunochromatography have been developed to improve the management of injured persons.

In currently proposed methods, the detection of the presence, or the measurement, of the concentration of antibodies specifically directed against the different vaccine antigens are conducted separately. Therefore several serum samples have to be taken which are analyzed separately, possibly in several laboratories each having analytical capacity for only one vaccine antigen or for a restricted number of vaccine antigens.

Some studies mention the determining of part of a person's vaccine status. For example, a seroprevalence study on the mumps, measles, rubella and chicken pox viruses was conducted in health care workers in Japan as part of a vaccine programme against these pathogens [Asari S. et al. Seroprevalence survey of measles, rubella, varicella and mumps antibodies in health care workers, and evaluation of a vaccination program in a tertiary care hospital in Japan; Am. J. Infect. Control. 2003, 31:157-[62]. In this survey, a single blood sample was taken, but the four serological tests, even though conducted by the same laboratory, were performed using different micro-titration plates on each of which an antigen of a single pathogenic agent was fixed, and following different methods. The ELISA methods used did not enable determination of the concentration of the specific antibodies tested. Also, these ELISA methods, having regard to the type of solid substrate used, implied sampling and analysis on relatively large volume samples of serum, namely the volume necessary to fill several microplate wells. This survey was not able to determine seroconversion against the specific antigens after vaccination in 20% of persons vaccinated against measles and rubella, and in 50% of persons vaccinated against mumps, attributable to poor sensitivity of the detection methods used. The different methods proposed for determining vaccine status often entail response times to obtain results that are longer than 24 hours and in general with poor sensitivity and hence poor reliability.

At the current time there exists no kit or automated, reproducible test with which it is possible to determine the vaccine status of a person by determining the titre of specific antibodies against the chief vaccine pathogens. Therefore there is currently no method and kit which, in a time lapse of less than 24 hours and on a single serum sample, allows a person's vaccine status to be determined i.e. the detection or determination of concentrations of specific IgG antibodies related to all currently available vaccines.

The aim of the present invention is to provide a technique for determining a person's vaccine status which is quick, easy and low-cost, which may in particular be used by any laboratory, by analysing a single serum sample to be tested, for the simultaneous determination of antibody concentration against a plurality of currently available vaccines, and using a relatively small sample volume and hence compatible with samples taken from children including infants.

More particularly, it must be possible for the diagnosis technique to be automated and reproduced in reliable manner, both regarding its performance and the preparation of the solid substrates used.

For this purpose, the present invention provides a method for the serological determination of a person's vaccine status by detecting and quantifying serum antibodies of IgG type specific to the vaccine antigens of a plurality of pathogenic agents of bacterial, viral, fungal or parasitic type, characterized in that it comprises detecting and quantifying a complex of immunological reactions between each said vaccine antigen and respectively each said IgG-type antibody specific to said vaccine antigen, possibly present in a sample of human serum to be tested, by conducting the steps of:
1. contacting one same said sample of serum to be tested with:
   one same solid substrate on which a plurality of said vaccine antigens are fixed corresponding to a plurality of different pathogenic agents, preferably at least 3, further preferably at least 4 vaccine antigens of different pathogenic agents, on different areas of the substrate,
   in the presence of at least one detection substance reacting by complexing with said specific IgG-type antibodies, and not reacting with said vaccine antigens, and
2. determining the concentration of said specific IgG-type antibodies by quantifying the complexes resulting from the reaction of at least one said detection substance with said IgG specific antibodies complexed with said vaccine antigens fixed on said solid substrate.

After washing the solid substrate, the presence of said specific antibodies is verified in the sample by detecting the signal of the labelling element of said detection substance at the fixing site of the vaccine antigen on the solid substrate corresponding to said specific antibody, insofar as said detection substance specifically reacts with said specific antibody and does not react with said vaccine antigen.

Typically, said detection substance is a secondary antibody of animal origin.

In one preferred embodiment, said detection substance comprises fluorescent labelling.

By “fluorescent labelling” is meant that the detection substance, in particular the detection secondary antibody, has been made fluorescent by coupling or complexing with a suitable fluorescent substance such as fluorescein isothiocyanate, namely a substance emitting radiation detectable after its illumination, each said fluorescent substance being characterized by the wavelength at which it is to be illuminated (excitation wavelength) and the wavelength of the radiation it emits (emission wavelength).

As fluorescent labelling substance, particular mention may be made of fluorescein, coumarin, cyanine, and analogs and derivatives of these substances known to persons skilled in the art.

Quantification is performed by comparing the fluorescent signal emitted by the reaction complex [antigen-specific antibody-detection substance] of the tested serum with a reference curve obtained by calibrating control sera containing a known concentration of antibodies to be detected.

When a fluorescent substance is used, the fluorescence associated with the tested sample is directly read on a suitable apparatus able to detect radiation at the emission wavelength and to quantify the same.

Said apparatus is known to those skilled in the art.

Fluorescent labelling is particularly advantageous when determining vaccine status, insofar as it is essential to obtain precise, reliable quantification of the concentration of said IgG-type specific antibodies, which can be obtained with fluorescent signals whose intensity is directly proportional to the quantity of fluorescent molecules emitting the signal.

By “vaccine antigen” is meant an antigen able to stimulate an immune response in the patient, causing the production of protective serum antibodies, i.e. an antibody binding to the pathogenic agent such that the body is thereby protected against the pathogenic effects of said agent.

Advantageously, it is determined whether the concentration of said specific antibodies reaches a given threshold on and after which said IgG-type specific antibody has a protective action, protecting against the disease determined by the pathogen.

In some cases, it is effectively possible to determine a concentration of specific antibodies imparting protection in nearly 100% of vaccinated persons, the determination of this concentration (or threshold) being made by sero-epidemiological studies on vast populations.

In one preferred embodiment, said vaccine antigens comprise at least 3, preferably from 3 to 12 vaccine antigens of pathogenic agents chosen from among the following viruses: mumps, rubella, measles, chicken pox, poliomyelitis, yellow fever, tick-borne encephalitis, hepatitis A, hepatitis B, and the following bacteria: Bordetella pertussis, tetanus and diphtheria.

Preferably, in the method of the invention, one same detection substance is used to detect the different antibodies specific to the different vaccine antigens.

Further preferably, and more particularly, a said detection substance is used which is an anti-IgG immunoglobulin, preferably a goat or chicken immunoglobulin.

The present invention therefore more particularly concerns a method to diagnosis the vaccine status of human infectious diseases, that is indirect and is based on the search and quantification in a patient's serum of antibodies specific to a vaccine agent of the infectious microbial agent, namely a bacteria, a virus, a parasite or a fungus responsible for a pathology (designated “microbe” below).

This method according to the invention is more particularly advantageous when at least one said vaccine antigen is a corpuscular microbial antigen consisting of a whole inactivated microbe or microbe fraction, in particular a vaccine antigen in the form of a viral suspension of whole, deactivated, live viruses.

The inventors have developed a technology by which this type of particulate or corpuscular antigen can be fixed onto the solid substrate by mere depositing and physical adsorption or physicochemical binding with the substrate, using a method for preparing a solid substrate according to the invention as is explained below. These particulate or corpuscular antigens have the advantage of being particularly clearly visible after depositing on the solid substrate, and in particular at the time of reading to detect any immunological reactions.

Advantageously therefore, as vaccine antigen, antigens consisting of whole, inactivated microbes are used or fractions or fragments of microbes comprising one or more antigens. It is effectively possible to fragment a microbe mechanically by mechanical agitation or sonication for example, or to fragment the microbe using an enzymatic process to obtain a fraction thereof conserving the antigens which withstand the serological reaction, subject of the present invention. The fractions so obtained are separated or purified from the other constituents of the microbe and its culture medium using a suitable method e.g. by centrifuging or filtering. In this case, the term antigenic fraction or purified antigen is used. The whole microbe, or any fraction of the microbe, is called a “particulate or corpuscular antigen” below when the whole microbe or one of its fractions cannot be placed in solution by dissolution, but solely in suspension in a suitable fluid.

The microbes or their fraction thus deposited are remarkably clearly visible as particles, individualized by microscope observation using an optical microscope or electronic microscope for example.

Technically, microbial serology consists of detecting in a patient's serum an antigen-antibody reaction in which the antigen is represented by all or a fraction of the infectious microbial agent to be detected, and the antibody is represented by human or animal immunoglobulins specific to said infectious microbial agent that are present in the patient's serum. It may be quantified by successively testing a series of increasing 2-fold or 10-fold dilutions of the patient's serum starting with a first dilution of 1/16 or 1/50 of the patient's serum.

Conventionally, one indirect serological diagnosis method more particularly comprises depositing the antigen on a solid substrate such as latex microbeads, in particular the detection technique by agglutination in which the latex microbeads are coated with the microbial antigen to be tested, and are agglutinated to each other by a patient's serum having specific antibodies, this agglutination being visible to the naked eye.

However, the detection technique by agglutination testing is both little practical to implement and little sensitive. In addition, it involves the use of high quantities of reagents and patient serum, and finally the reading of results cannot be automated.

According to the method of the invention, the antigen is therefore deposited on a solid substrate of glass slide type or a titration microplate, to implement detection techniques by immunodetection, preferably immunofluorescence. These detection techniques are well known to those skilled in the art and comprise the successive steps of:
1. decomplementing the serum by heating to 56° C. for 30 minutes,
2. contacting the antigen, corresponding to the infectious microbial agent fixed on the solid substrate, with the patient serum then incubating under conditions of time, temperature, hygrometry, mechanical agitation and ionic strength of the medium allowing the antigen-antibody reaction,
3. careful, thorough washing to remove the excess patient serum that is not fixed to the solid substrate,
4. applying a secondary detection antibody which is an animal immunoglobulin directed against the immunoglobulins of the patient species under consideration, e.g. for a human patient, a goat anti-human immunoglobulin conjugated with a fluorochrome substance, generally fluorescein isothiocyanate, and incubation under conditions of time, temperature, hygrometry, mechanical agitation and ionic strength of the medium allowing the antigen-antibody reaction,
5. thorough, careful washing to remove the excess, non-fixed labelled immunoglobulin,
6. detecting a reaction by reading results, using suitable apparatus for the labeller used, such as a fluorescence microscope or a microarray for the indirect immunofluorescence technique, known to those skilled in the art.

Advantageously, said detection substance used is a detection secondary antibody, an animal immunoglobulin directed against the immunoglobulins of the patient species under consideration, e.g. for a human patient, a goat anti-human IgG antibody which reacts with specific class G immunoglobulins complexed with said vaccine antigen.

No serological test currently marketed systematically includes control of the addition and reactive value (or immunological reactivity) of the anti-IgG detection secondary antibodies used, and another original purpose of the present invention is therefore to provide a test comprising this reactivity control.

Preferably, in the method of the invention, the presence and reactivity of said first detection substance is previously controlled by performing the steps of:
contacting said sample to be tested with one same said solid substrate, on which a first control antigen has also been fixed which is non-specific class G immunoglobulin, in the presence of a solution containing a said first detection substance, and
verifying whether said detection substance has reacted with said first control antigen fixed on said solid substrate.

The specificity of the infectious antigen/serum antibody reaction determines the specificity and positive predictive value of the serological test based on this reaction, and any fixing of a non-specific antibody on the microbial antigen limits the specificity and predictive value of the serological test. The presence in the serum to be tested of antinuclear antibodies is a source of non-specific fixing of antibodies on the microbial antigen as explained below.

The presence in the patient serum of antinuclear antibodies limits the specificity of the microbial antigen/serum antibody reaction. Antinuclear antibodies are IgG-type antibodies directed non-specifically against the group consisting of the DNA and nuclear proteins of the chromosome of eukaryote cells and microbes, called histones. These antinuclear antibodies therefore fix non-specifically onto any eukaryote cell, including fungi and parasites and onto any microbe, bacterium, DNA virus, parasite or fungus. This phenomenon causes a non-specific, positive reaction during serological tests using specific IgG detection of a bacterium, DNA virus, fungus or whole parasite, or comprising DNA/histone complexes as microbial antigen, using anti-IgG detection antibodies, in a serum containing antinuclear antibodies.

The detection of antinuclear antibodies in serum to be tested, using an immunofluorescence immunodetection technique, has been described [Fritzler M. J. In: Manual of Clinical Laboratory Immunology, Fourth edition, Rose N. R., Conway de Macario E., Fahey J. L., Friedman H., Penn G. M., (eds) American Society for Microbiology, Washington D.C. 1992, pp. 724-729] using mammalian cells including human cells such as Hep-2 cells as antigen. Under these conditions, the prevalence of antinuclear antibodies detected in pure serum is 2% in the general population, 20% among relatives of patients suffering from rheumatoid polyarthritis, and 75% in elderly persons with no apparent clinical pathology ([McCarty G. A. et al Antinuclear antibodies: Contemporary techniques and clinical applications to connective tissue diseases. Oxford University Press, New York, [984]. In this method, the cells consist of a mat of confluent cells deposited and “read” manually. This deposit of confluent cells is too viscous to be automated by depositing with a syringe-type spotter, and reading cannot be automated since solely manual depositing enables a large quantity of cells to be deposited in order to guarantee sufficient detection of the cell/antinuclear antibody reaction. In these publications, the need is not mentioned to carry out systematic control of the presence of antinuclear antibodies in immunodetection tests of microbial antigens. In addition, detection with confluent cells deposited manually on a solid substrate is not applicable to routine laboratory tests.

There is therefore no method published to date to control or remove antinuclear antibodies before conducting a serological test to diagnose infectious diseases, in order to guarantee that results do not contain any false positive reaction, and which can be given routine laboratory application. This is why no serology protocol published in reference manuals, and no marketed serological tests systematically include the prior screening of antinuclear antibodies before serological testing or interpreting.

In practice, to date, in routine laboratory immunodetection tests, it is only possible at the most to detect false positives due to the presence of antinuclear antibodies by conducting tests on a plurality of microbial antigens whose concomitant presence is improbable. But it will easily be understood that this type of verification considerably increases costs in terms of equipment, labour and time consumption.

For this purpose, the present invention provides a method in which the possible presence of an antinuclear antibody is controlled in said sample to be tested, by:
contacting said sample to be tested with:
   a solid substrate on which a second control antigen has been fixed comprising DNA/histone complexes, preferably comprising nuclei of nucleated cells of the patient species, further preferably all or part of continuous line cells originating from the patient species, and
   in the presence of said detection substance consisting of a labelling antibody which only reacts with said class G immunoglobulin of the patient species; and
verifying whether said second control antigen fixed on the solid substrate reacts with said detection substance.

In the case of a human patient, it is possible in particular to use non-confluent human fibroblast cells in suspension, in particular HL60 cells.

If the sample to be tested contains antinuclear antibodies (which are IgGs, human IgGs in particular) these may react with said second antigen (Ag₂) and be detected by said detection substance (Ac₁ anti-IgG*) since this detection substance is a substance reacting with the IgGs of the patient species, human particular, and forming the complex (S-Ag₂-IgG anti Ag₂-Ac₁ anti IgG*).

Once the absence of any antinuclear antibodies has been established, the detection of a reaction of said detection substance with said microbial vaccine antigen is proof of the presence of IgG specific to said microbial vaccine antigen and of the formation of a complex (S-Agvac-IgG antiAgvac-Ac₁ anti IgG*) and not of a false positive complex resulting from the reaction of the antinuclear antibodies with the microbial antigen giving the complex (S-Agvac-Ac antinucl-Ac₁, anti IgG*).

Further preferably, the reactivity of said detection substance is verified that is added to said serum sample to be tested, before verifying the absence of antinuclear antibodies in said sample.

If said detection substance is reactive, the following complex should be detected (S-IgG₁-Ac₁ anti IgG*) with said first antigen (IgG₁). In this case, the absence of a reaction of said detection substance with said second antigen is proof of the absence of antinuclear antibodies.

More particularly, depositing the IgG of the patient species, human in particular and γ-specific (specific to the gamma chain of immunoglobulins of the patient species, human in particular) as said first antigen allows positive control of the addition of the anti-IgG conjugate immunoglobulin during the serological reaction, and its immunological reactivity (qualitative aspect). This conjugate antibody will also fix itself onto the IgG deposit which will therefore necessarily be recognized during the detection phase of IgGs specific to the serological reaction.

With the present invention it is therefore possible to systematically detect antinuclear antibodies in a serum used for serological diagnosis by indirect immunofluorescence, after auto-spotting class G (IgG) immunoglobulins of the patient species, human in particular, and nucleated cells of the patient species, human in particular, on the substrate of the serological reaction. Similarly, with the automated spotting of immunoglobulins of the patient species, in particular human IgGs, it is possible to control the presence of the anti-IgG secondary antibodies of the patient species, human in particular, that are used for testing.

A frequent error when conducting serological tests, in particular batch serological testing conducted on a large number of sera to be tested, is due to faulty addition of the sera to be tested, in particular by pipetting. These errors occur in particular during the steps involving displacement of the sample to be tested, by pipetting in particular, some recipients in particular those containing the solid substrate on which the antigen to be detected is deposited, may inadvertently not be filled with the serum of the patient species, human in particular, to be tested. It is known that serum pipetting carries an error risk of 1% due to a purely technical problem through non-pipetting by the pipette, or to a human error through inadvertent failed pipetting.

These errors require the introduction of controls when carrying out the reaction. The systematic incorporation, during each new handling, of a negative control serum, i.e. not containing antibodies specific to the antigen to be tested, will allow positive reactions to be interpreted. Similarly, the incorporation of a positive control serum i.e. containing the antibody specific to the antigen to be tested at a known concentration, allows the quality of the antigen and conjugate immunoglobulin to be verified.

However, at the present time no reliable control exists to verify that the serum to be tested has effectively been added to the serological test. Yet if by inadvertence the serum to be tested is not added to the serological test, the bacterial antigen/serum antibody reaction will certainly not take place and the test will falsely be interpreted as negative (false-negative). In the present invention, advantage is drawn from the fact that protein A of Staphylococcus aureus has an affinity for sera of animal origin, in particular of horse, bovine, porcine, rabbit, guinea-pig, mouse origin; to a lesser extent, of hamsters, rats and sheep. On the other hand, chick and goat serum do not react with protein A. Protein A is a polypeptide of 42 kDa and is one of the constituents of the wall of Staphylococcus aureus strains, similar but different proteins are characterized on the surface of bacteria of genus Streptococcus [Langone J. J. Adv. Immunol. 1982, 32:157-251]. This property of protein A of Staphylococcus aureus has already been used in a serological test in man for the serological diagnosis of infectious endocarditis [Rolain J M, Lecam C. Raoult D. Simplified serological diagnosis of endocarditis due to Coxiella burnetii and Bartonella; Clin. Diag. Lab. Immunol. 2003. 2003; 10:1147-8].

The present invention therefore comprises the addition of a control to the serum to be tested during the serological reactions.

For this purpose, it is controlled that said tested sample effectively contains a serum of the patient species, by detecting whether immunoglobulins of the patient species react with a third control antigen containing protein A of a Staphylococcus aureus bacterium, preferably by contacting said sample with a same said solid substrate on which said third control antigen is fixed, in the presence of a same said detection substance which is a substance reacting with an immunoglobulin of the patent species and not with said third control antigen, preferably an anti-immunoglobulin antibody of the patient species, and not reacting with protein A.

Insofar as protein A reacts non-specifically with animal and human immunoglobulins, even in major infectious pathologies, it is possible to use this protein A as positive control, to control the addition of a serum of the patient species, human in particular, to the sample to be tested.

In one advantageous embodiment, said third control antigen is a whole Staphylococcus aureus bacterium containing protein A. More particularly, use may be made of the Staphylococcus aureus bacteria deposited in public collections such as the bacteria deposited with A.T.C.C. under No 29213 and the C.N.C.M. at Institut Pasteur (France) under No 65.8T, as described in the publication mentioned above [Rolain J M, Lecam C, Raoult D. Simplified serological diagnosis of endocarditis due to Coxiella burnetii and Bartonella. Clin. Diag. Lab. Immunol. 2003; 10:1147-8].

Also, apart form type strains, any bacterial strain identified as Staphylococcus aureus may be used as said third control antigen.

Preferably, said detection substance is an animal immunoglobulin, further preferably a goat or chicken immunoglobulin.

Advantageously, the successive steps are conducted of:
controlling the presence of a serum in the sample to be tested, in the presence of said detection substance, and
controlling the presence of antinuclear antibodies in the sample to be tested.

Further advantageously, said class G immunoglobulin of the patient species specific to said vaccine antigen present in the sample to be tested is detected and optionally assayed by automated reading of the intensity of the signal emitted by said labelling element, using reading apparatus suitable for said signal labelled by said element.

As labelling element for said detection substances, a radioactive labeller may also be used, although fluorescent labelling remains preferred as mentioned previously.

The expression “radioactive labelling” means that the antibody carries a radioactive isotope allowing its assay by counting the radioactivity associated therewith, the isotope possibly being carried either on an element of the structure of the antibody, e.g. constituent tyrosine residues, or on a suitable radical fixed to it.

If a radioactive probe is used, e.g. iodine 125, the radioactivity associated with the tested sample is counted in a gamma counter using any appropriate method e.g. after solubilizing the cells with an alkaline solution (a sodium hydroxide solution for example) and collecting the solution containing the radioactivity with an absorbent buffer.

In one embodiment, a protocol for successive reading of controls is the following:

1) It is verified that said third control antigen containing protein A reacts with a said detection substance. If not, the test is stopped i.e. this sample is not taken into account.

2) If said first control antigen (IgG₁) does not react with said detection substance (Ac₁ anti IgG*) said detection substance is not present or is not reactive. The result of the test concerning the detection of IgGs specific to the microbial antigen is not taken into account.

3) If said first control antigen (IgG₁) reacts with the detection substance, the test can be continued i.e. the results can be taken into account subject to the following verifications concerning reactions with the control antigens.

4) If said second control antigen containing a DNA/histone complex reacts with said detection substance, antinuclear antibodies are present and the detection and quantification tests of specific IgGS are not taken into account.

5) If said second antigen does not react, and if said detection substance is present and reactive, there are no antinuclear antibodies and the test can be continued subject to the following verification.

To summarize, the result of the reaction with said microbial vaccine antigen is only taken into account if the following accumulative conditions are met:
said first control antigen (IgG) reacts with said detection substance, and
said second control antigen (DNA) does not react, and
said third control antigen (protein A) reacts with said detection substance.

In one preferred embodiment, for each detection and, when applicable each quantification of a said vaccine antigen, the following measurements are made:

1—a first measurement of a first value representing the quantity of a first labelling element, preferably the first value of the intensity of a signal emitted by said first fluorescent labelling element, said first labelling element fixing non-specifically to any protein in the deposit area of said vaccine antigen, and

2—a second measurement of a second value representing the quantity of a second labelling element, different to said first labelling element and emitting a different signal, preferably a second value of the intensity of the signal emitted by this second fluorescent labelling element at a different excitation wavelength to that of said first fluorescent labelling element, said second labelling element being the labelling element of said detection substance of said vaccine antigen, in the deposit area of said antigen, and

3—the ratio between said first and second values is calculated, and

4—the value of said ratio is compared with the value of a reference ratio obtained with a collection of positive and negative reference sera, thereby allowing the determination by comparison of whether or not there is a need to vaccinate the person for said vaccine antigen, depending on the value of the ratio of said first and second values.

The present invention also provides a diagnosis kit which can be used to implement the method of the invention, characterized in that it contains:
one said same solid substrate on which at least one said plurality of vaccine antigens is fixed, and optionally at least one said control antigen, and
reagents such as a said detection substance and, if necessary, reagents to detect said labelling element.

Preferably the kit comprises:
one same said solid substrate on which at least one said corpuscular vaccine antigen and said first, second and third control antigens are fixed, and
one same said detection substance.

Advantageously, in the methods and diagnosis kit of the invention, as solid substrate a glass or plastic slide is used, or a well of a flat-bottomed microtitration plate in glass or plastic.

To conduct the determination of vaccine status according to the present invention, it is possible to sample serum by vein puncture with a needle or by sampling capillary blood, in particular from the ear lobe, finger pad, heel pad, coupled with collection on a disc of filter paper or preferably directly in a buffer allowing elution of the serum.

According to another advantageous characteristic of the present invention, after sampling capillary blood in a capillary tube allowing a known volume of whole blood to be collected, said sample is placed in a bottle containing a known volume of elution buffer. This original collecting method has the advantage of being rapid (collection, elution and dilution being performed in one minute). The fact that the serum is diluted to a known concentration, 1/20 to 1/100 in particular, provides a safety barrier against the risk of blood exposure accidents such as the transmission of viruses and other blood pathogens, for the person taking the sample.

Also, the present invention allows the determination of a person's vaccine status against several vaccine antigens simultaneously, on a single serum sample taken by capillary puncture and in a time lapse of less than 2 hours.

Advantageously, a determined volume of whole blood is collected using a capillary tube in a bottle containing a determined volume of a buffer allowing elution of the serum, the serum then preferably being diluted to a determined concentration, preferably 1/100 to 1/20, and a determined volume of the diluted serum is deposited on the different deposit areas of said control antigens and vaccine antigens on said solid substrate.

Irrespective of the method for collecting blood, whether “vein puncture” or “capillary puncture” the serum must initially be separated from the blood cells. With capillary puncture, this separation is made by eluting with an elution buffer and it is the product of this elution which is contacted with the slide.

Advantageously therefore, the kit of the invention comprises a bottle containing a determined volume of an elution buffer to collect a determined volume of a serum sample to be tested.

Methods and diagnosis kits are known which use a solid substrate on which soluble proteins are covalently fixed, but these covalent chemical couplings are complex and costly to perform. The non-covalent fixing of soluble proteins or particulate antigens has been proposed on a solid substrate by physical or physicochemical adsorption on the substrate, for immunodetection test protocols on a solid substrate, but the stability of the fixing is insufficient. One difficulty lies in the fact that it is necessary to thoroughly wash the solid substrate previously to eliminate residues of labelling elements which may interfere with the reading of results, but these washings make the physical adsorption of the substances on the solid substrate too unstable. Another difficulty is that it is not possible with automated spotters to deposit corpuscular antigens as such, whether they are cells or whole or partial bacteria as mentioned above.

A further purpose of the present invention is therefore to provide a reliable test for the immunodetection of microbial vaccine antigens, in particular particulate viral vaccine antigens, using a simple method and tooling which can be given routine application in a laboratory, in particular for the conducting of tests in series.

A further purpose of the present invention is to provide a method for preparing a solid substrate allowing automated spotting on the substrate of control antigens or particulate or corpuscular vaccine antigens, and the automated reading of the results of the immunological reaction of said antigens fixed on the solid substrate, in a patient serum test involving a microbial serology reaction of specific IgGs by adsorption with the particulate or corpuscular vaccine antigen or control antigen deposited on the solid substrate.

The present invention provides a method for preparing a solid substrate which can be used in a method or kit of the invention, characterized in that on said solid substrate a plurality of said vaccine antigens are deposited and fixed comprising at least one corpuscular vaccine antigen in the form of a suspension of whole microbes or fractions of microbes, in particular viruses or bacteria, preferably and optionally a vaccine antigen in the form of a whole, deactivated living virus, and optionally of said control antigens in the form of a suspension of corpuscles of non-confluent cells or whole bacteria or fractions of cells or bacteria, said corpuscular antigens preferably being deposited by an automated spotter comprising a syringe in particular.

Preferably, said vaccine corpuscular antigens and/or control antigens are associated with a dye, preferably a fluorescent dye, in suspension form at a concentration allowing their visualization after depositing by means of said dye to verify the fixing of said antigens on the solid substrate.

According to the present invention, the inventors, after numerous fruitless attempts, have been able to determine the conditions for the automated depositing of corpuscular microbial vaccine antigens (inactivated whole microbes or fractions of whole inactivated microbes) in suspension in a depositing medium. At the present time, automated spotters are solely used to deposit homogenous solutions of one or more molecules on a solid substrate. For this purpose, the concentration of these corpuscular antigens is calibrated before deposit by counting the inactivated microbial particles by “fluorescence activated cell sorting” (FACS-scann) then deposited by automated spotter on a solid substrate. Determining these calibrated, automated deposits entails the determination by tests of the optimal concentration for each of the antigens tested, infra-optimal concentrations giving undetectable deposits, supra-optimal concentrations leading to sedimentation during deposit of corpuscular antigens of high density and micrometric size such as whole or fractioned bacteria, and hence a substantial variation in the quantity of deposited antigen. Finally, the deposits of corpuscular antigens containing microbial DNA (bacterium, virus, parasite or microscopic fungus) are calibrated by applying a dye preferably a fluorescent dye, in particular a molecule such as AMCA, which fixes non specifically to the proteins contained in the antigen, or an intercalating molecule which fixes non-specifically to DNA by intercalation in the double helix. This latter method is preferably used to label cells used as controls on the slides. The excitation and emission wavelengths are advantageously chosen in relation to the those used by the fluorochrome labelling the detection immunoglobulins. This fluorescent labelling, non-specific to the antigens, can be made before or after automated spotting. The fluorescent labeller is advantageously chosen for its stability to daylight.

More particularly, in the method for preparing a solid substrate according to the invention, the control antigens in cell suspension form are calibrated at a concentration of 10⁷ to 10⁹ cells/ml, the suspensions of bacteria or fractions of bacteria at a concentration of 10⁷ to 10⁹ particles/ml, and the suspensions of whole viruses at a concentration of 10⁹ to 10¹⁰ particles/ml.

Advantageously, said control antigens and corpuscular vaccine antigens are deposited in a mixture with a protein binder, stabilizing their fixation onto said solid substrate.

More particularly, said protein binder is chosen from among the complex organic mixture formed of egg yolk, gelatine, bovine serum albumin or a non-human polyclonal IgG, preferably goat.

These protein binders acts as a biological glue for said antigen on the solid substrate.

The different binders were tested on a glass slide, and the optimal concentrations of use were determined. In particular bovine serum albumin may be used at a final concentration (volume/volume) of 1 to 5%, a suspension of egg yolk at a final concentration of 1 to 10%, and said caprine IgG at a final concentration of 25 to 75%.

The inventors observed, during the different tests conducted, that some antigens bound by egg or bovine albumin were washed off during the washing steps, but the human IgG immunoglobulins added as controls invariably remained fixed to the glass slide. The inventors deduced that class G immunoglobulins fixed themselves to the glass slide in such manner that they withstood the washing steps, and considered the possibility that these IgGs could also be used under suitable conditions to fix certain vaccine antigens. The inventors therefore used IgGs of a species other than man, to avoid interference with the serological tests, and the authors thus determined the remarkable properties of goat IgG as biological glue allowing the fixing of particulate antigens, in particular particulate or corpuscular vaccine antigens.

Preferably, and more particularly, said corpuscular vaccine antigen is deposited on said solid substrate consisting of a glass slide, in a mixture with an immunoglobulin of goat polyclonal IgG type.

A further subject of the invention is a method for preparing a solid substrate on which a plurality of said vaccine antigens is fixed, and optionally said first, second and third control antigens, to allow detection by automated reading and said detection substance, which can be used in a method of the invention or a kit of the invention, characterized in that prior washing of said solid substrate is performed with a solution of an ethanol/acetone mixture, preferably 50/50, then said antigens are deposited on said solid substrate by physical adsorption and their fixing stabilized by treatment with alcohol, preferably methanol or ethanol, which alcohol is then removed, and preferably the fixing of said antigens is verified by staining, in particular by fluorescent labelling non-specific to proteins or DNA as explained above.

This pre-washing solution allows the substrate to be cleaned of all traces of detection substance or other residual, and in particular to eliminate all fluorescence, while maintaining the substrate's physical adsorption property for said antigens that are subsequently deposited.

Stabilization treatment with alcohol allows stabilization of fixing by physical adsorption, both of the proteins such as IgGS and of the particulate antigens.

Determining suitable prior washing of the solid substrate, the glass slide in particular, necessitated numerous tests. The objective of this treatment is to clean this substrate thoroughly to eliminate fluorescent artefacts, while preserving subsequent fixing of the antigens in a manner compatible with their conservation mode but also by preserving if not the integrity at least the immunological reactivity of the antigens. It was shown, after numerous fruitless attempts, that rinsing and cleaning the slides in an aqueous phase did not permit subsequent fixing of the antigens to be deposited; the same applied to rinsing with surfactant molecules such as Tween 20. Cleaning with alcohols was not sufficient to remove most of the fluorescent artefacts. It was therefore after multiple tests that a protocol could be optimized for cleaning the solid substrate, the glass slide in particular, using a mixture of 50% ethanol-50% acetone followed by air drying.

However, even in this case, it remains advantageous to complete fixing by physical adsorption using a cross-linking treatment, in particular chemical treatment with a bi-functional agent for covalent coupling such as glutaraldehyde or activated diacid derivatives, in particular succinic acid, known to persons skilled in the art, to ensure covalent bridging between said control antigens and microbial antigens with the solid substrate.

Other characteristics and advantages of the present invention will become apparent in the light of the following examples.

### EXAMPLE 1

### Depositing Said Second and Third Control Antigens on the Solid Substrate

HL60 cells (No ATCC CCL 240) are continuous line human fibroblast cells used to detect antinuclear antibodies. After culture and production following usual protocols, the concentration of the HL60 cells was quantified using a cell counter (Microcytes® BPC/Yeast, Bio DETECT AS, Oslo Norway) and this concentration was calibrated to 10⁸ cells/mL in a sterile BPS buffer, pH=7.4, to obtain a suspension of non-confluent cells which could be auto-spotted.

Staphylococcus aureus (ATTC No 29213) was cultured on agar with 5% sheep blood, then harvested in sterile PBS buffer containing 0.1% sodium azide. The inoculum was measured using the same cell counter and calibrated to 10⁹ bacteria/mL which is the optimum concentration having regard to constraints of non-sedimentation during deposit and depositing of a sufficient quantity of staphylococcus particles, then stored by freezing to −80° C.

These HL60 cells and Staphylococcus aureus bacteria were deposited at a concentration of 10⁹ CFU/mL determined by FACS-scan (Microcytes®) on glass slides (Reference LLR2-45, CML, Nemours, France). The cells and bacteria were deposited on the solid substrate using a spotter (Arrayer 427™, Affymetrix, MWG Biotech SA, Courtaboeuf, France). The deposits were air dried for 30 minutes in the spotter chamber, then fixed in a bath of 100% methanol for 10 minutes, then dried again in open air. The efficacy of spotter depositing on the slides, after fixing with ethanol and after the baths required by the following indirect immunofluorescence reaction, was successively verified by staining with Hoescht 333-42 fluorescent dye (intercalating molecule which intercalates DNA) which is excited at 350 nanometres and which emits at 460 nanometres (Molecular Probes).

### EXAMPLE 2

### Depositing Said First Control Antigen (IgG) on a Solid Substrate

Human, class G, γ-specific immunoglobulins (IgG) (Serotec, Cergy Saint-Christophe, France) diluted to a concentration of 5 mg/mL to obtain fully homogeneous spots were deposited using a spotter (model 427, Arrayer, Affymetrix, Inc., CA) on a solid substrate consisting of a glass slide (Reference LLR2-45, CML, Nemours, France).

The deposits were made by transfer of the antigen suspension from a well of a microtitration plate containing 25 μL suspension, in a volume of 1 mL, deposited at 25° C. and 55% humidity in the spotter chamber. These conditions were controlled with a thermo-hygrometer. The deposits of size 200 μm were air dried for 30 minutes at 37° C. in the spotter chamber, then fixed in a bath of 100% ethanol for 10 minutes, then dried again in open air. The efficacy of automated spotting, then of fixing in ethanol after the baths required by the indirect immunofluorescence reaction, was verified using the indirect immunofluorescence technique.

10 human sera were each deposited on a IgG deposit, each in three dilutions, 1:32, 1:64 and 1:128. After a first washing, an indirect immunofluorescence reaction was performed using a goat immunoglobulin (Reference A-21216, Molecular Probes, Eugene, USA) as anti-human IgG secondary antibody coupled to Alexa 488 which is a fluorescent molecule excited at 488 nanometres and emitting at 540 nanometres, and using the same anti-IgG immunoglobulin coupled to Alexa 594 (A-21216, Molecular Probes, Eugene, USA) which is a fluorescent molecule excited at 594 nanometres and emitting at 640 nanometres in a second experiment. Reading of the reaction was made under a fluorescence microscope and showed fluorescent detection in all sera in the form of a very shiny spot, for each of the 3 tested dilutions. This example illustrates that it is possible to achieve automated spotting of human IgGs on a solid substrate under conditions compatible with the performing of an indirect immunofluorescence reaction.

### EXAMPLE 3

### Determining a Person's Vaccine Status

A glass slide was developed to determine the vaccine status of a person, comprising a total of 8 antigen deposits including 3 control deposits and 5 vaccine antigen deposits on one same slide, divided into two rows. Conditions for depositing and use of the 3 control deposits containing Staphylococcus aureus, IgG and HL-60 cells, were described in examples 1 and 2 above.

The deposits of vaccine antigens were made in the form of a viral suspension of whole, inactivated, living viruses deposited at a concentration of 10⁹ or 10¹⁰ particles/ml in a mixture with a caprine IgG at a concentration of 25 to 75% (volume/volume) and under the following conditions:

(1) Rubella antigen: the strain used was hpv-77 (Microbix Biosystems Inc., Toronto, Ontario, Canada) supplied in the form of an inactivated viral suspension at a protein concentration of 0.51 mg/ml. This antigen was concentrated 10 times by centrifuging, before being deposited in a volume of 45 μL antigen and 5 μL goat IgG immunoglobulins at a concentration of 10 mg/ml (Reference 15256, Sigma, Saint-Quentin Fallavier, France) stained with 2 μL amino methyl coumarin acetyl (AMCA) (Molecular Probes, Interchim, Montluçon, France).

AMCA fixes non-specifically to the proteins of this antigen and therefore allows verification of the depositing of the antigen on the solid substrate by physical adsorption. It is excited at the same wavelength of 350 nm as the HOESCHT 333-42 dye.

(2) Measles antigen: the strain used was Edmonston (Microbix Biosystems Inc.) supplied in the form of a suspension of inactivated viral particles at a protein concentration of 1.95 mg/ml, which was concentrated 5 times by centrifuging before being deposited, in a volume of 45 μL antigen and 5 μL goat IgG immunoglobulin stained with AMCA.

(3) Mumps antigen: the strain used was Enders (Microbix Biosystems Inc.) at a protein concentration of 1.8 μg/ml and deposited in a volume of 45 μL antigen and 5 μL goat IgG immunoglobulin labelled with AMCA.

(4) Diphtheria toxoid (Reference FA 150934, Aventis, Pasteur Vaccins) at a protein concentration of 18.9 mg/ml and concentrated 10 times before being deposited in a volume of 45 μL antigen and 5 μL goat IgG immunoglobulin labelled with AMCA.

(5) Tetanus toxoid (Reference J001, Aventis Pasteur Vaccins) at a concentration of 80 IU/mL and deposited pure in a volume of 45 μL antigen and 5 μL goat IgG immunoglobulin labelled with AMCA.

The antigen deposits were made in a volume of 1 nl using an Affymetrix® spotter, model Arrayer 427. After drying, the slides thus prepared were used as substrate for an indirect immunofluorescence reaction for the detection of IgGs specific to vaccine antigens in the serum of individuals, in accordance with the following protocol:

1. In a first step, 5 μL of serum sampled by vein puncture or capillary puncture, are contacted and incubated with the slide at the levels of the different vaccine antigens and control antigens, in an automated incubator.

2. In a second step, the incubator rinses the slide to remove the tested serum and incubates the slide with the anti-human IgG detection antibody conjugated with fluorescein which is excited at a wavelength of 488 nm (reference Star 106F, Serotec, France).

3. In a third step the incubator rinses the slide to remove the conjugate detection antibody, and dries the slide.

4. In a fourth step, the slide this treated is removed from the automated incubator and placed in the reading chamber of an automatic fluorescence reader. This reader successively carries out two readings, one at 350 nm which is the emission wavelength of the AMCA dye, and the second reading at 488 nm which is the emission wavelength of the fluorescein of the detection antibody.

5. In a fifth step, this data is transmitted to a software programme for conversion into fluorescence intensity at 350 nm and 488 nm for each of the deposits.

6. In a sixth step, the software analyses the fluorescence levels of the controls and successively verifies:
the presence of fluorescence for the Staphylococcus aureus deposit, to verify the presence of the serum to be tested;
the presence of fluorescence for the IgG deposit, to verify the quality of the reaction involving the conjugate detection antibody;
the absence of fluorescence for the deposit of HL60 cells, to verify the presence or absence of antinuclear antibodies in the serum to be tested.

7. In a seventh step, the software calculates the fluorescence ratio 488/350 for each of the deposits of vaccine antigens, and for each vaccine antigen deposit compares this value with a ratio curve previously determined for each vaccine antigen using positive control sera containing specific antibodies at a known concentration by a reference method, and using negative sera not containing specific antibodies detectable by a reference method.

The fluorescence at 350 nn results from excitation of non-specific labellers which fix non-specifically either to DNA or to the proteins of the vaccine antigens. The quantity of fluorescence emitted by the antigen deposits at 350 nn is proportional to the quantity of antigens effectively present in the deposit, which means that this fluorescence at 350 nn is a measurement dependent upon the quantity of antigens present in the deposit. The quantity of fluorescence at 350 nn is used by the software programme:
firstly to identify the topographical position of the antigen deposits on the slide, and
secondly to quantify their exact surface area and hence their contours so as not to incorporate fluorescence artefacts lying outside these spots, and
finally, to weight fluorescence at 488 nn. This second wavelength of 488 nn results from excitation of the labeller of the detection G immunoglobulin. The quantity of fluorescence at 488 nn is related to the quantity of specific G immunoglobulin present in the spot of patient serum to be tested. Fluorescence at 488 nn (and hence fixing of the G immmunoglobulins) is dependent on the quantity of antigens deposited. If very little antigen is deposited, the quantity of fixed, specific immunoglobulin will be small and hence the level of fluorescence at 488 nn will be low. This is why the quantity of fluorescence at 488 nn is weighted, for each of the spots of vaccine antigens, with the quantity of fluorescence at 350 nn and the ratio of fluorescences expressing the quantity of specific IgGs present in the tested serum.

8. In a final step, the software interprets the fluorescence ratios 488/350 measured on the tested sera by comparing them with the measurements obtained for a collection of positive and negative reference sera for each one, used to determine reference curves, all this data being compiled to indicate the list of vaccine antigens against which the tested serum contains specific antibodies, thereby giving the vaccine status of the person.

In this example therefore four sera were tested, taken from four different patients, to search the presence of class IgG antibodies against the vaccine antigens of measles, rubella, mumps, diphtheria and tetanus. The threshold value of the fluorescence ratio was determined for each of these 5 vaccine antigens.

Tables 1A, 1B, 1C and 1D show the fluorescence ratios for each of the 4 tested sera respectively.

From Tables 1A to 1C it can be seen that:

serum no 1 is:
positive for the presence of diphtheria anti-toxoid antibodies (fluorescence ratio >1, threshold value),
positive for the presence of tetanus anti-toxoid antibodies (fluorescence ratio >0.05, threshold value),
negative for the presence of anti-rubella antibodies (fluorescence ratio <0.1, threshold value),
positive for the presence of anti-measles antibodies (fluorescence ratio >0.1, threshold value), and
positive for the presence of anti-mumps antibodies (fluorescence ratio >0.11, threshold value).

serum no 2 is:
positive for the presence of diphtheria anti-toxoid antibodies (fluorescence ratio >1, threshold value),
positive for the presence of tetanus anti-toxoid antibodies (fluorescence ratio >0.05, threshold value),
positive for the presence of anti-rubella antibodies (fluorescence ratio >0.1, threshold value),
positive for the presence of anti-measles antibodies (fluorescence ratio >0.1, threshold value), and
positive for the presence of anti-mumps antibodies (fluorescence ratio >0.11, threshold value).
serum no 3 is:
negative for the presence of diptheria anti-toxoid antibodies (fluorescence ratio <1, threshold value),
negative for the presence of tetanus anti-toxoid antibodies (fluorescence ratio <0.05, threshold value),
negative for the presence of anti-rubella antibodies (fluorescence ratio <0.1, threshold value),
positive for the presence of anti-measles antibodies (fluorescence ratio >0.1, threshold value), and
negative for the presence of anti-mumps antibodies (fluorescence ratio <0.11, threshold value.

serum no 4 is:
negative for the presence of diptheria anti-toxoid antibodies (fluorescence ratio <1, threshold value),
negative for the presence of tetanus anti-toxoid antibodies (fluorescence ratio <0.05, threshold value),
positive for the presence of anti-rubella antibodies (fluorescence ratio >0.1, threshold value),
negative for the presence of anti-measles antibodies (fluorescence ratio <0.1, threshold value), and
negative for the presence of anti-mumps antibodies (fluorescence ratio of <0.11, threshold value.

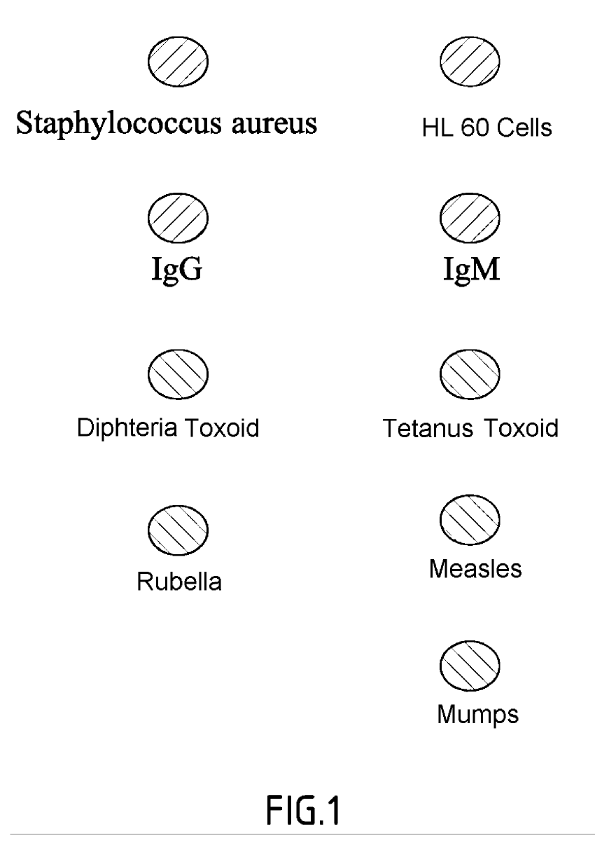

FIG. 1 is a diagram showing the layout of the deposits of antigens on the vaccine slide.

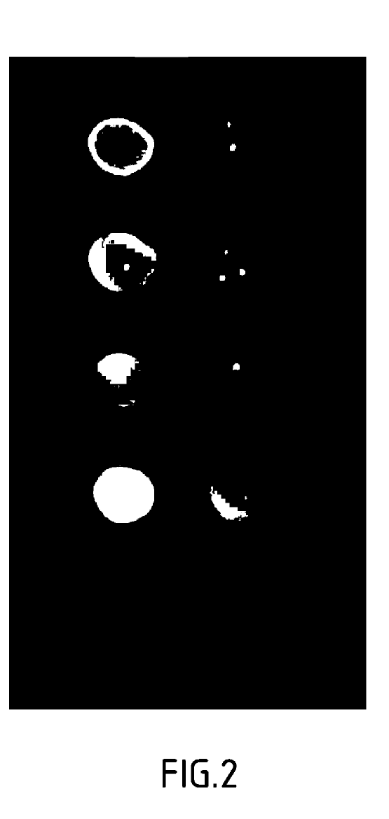

FIG. 2 is the picture of this slide after incubation with the 4 sera cited above, obtained at 350 nm (non-specific fluorescent staining after non-specific fluorescent labelling of proteins with AMCA and of DNA with Hoescht 332-42 dye). This image can be used to control the presence of each of the deposits of control and vaccine antigens.

FIGS. 1 and 2 show an IgM spot (deposit area) which is not used for the tests conducted.

TABLE 1A
Serum N^{o} 1

| | | | | | Ratio | Fluores- |
|---|---|---|---|---|---|---|
| | | | Surface | Fluores- | F350/ | cence |
| N^{o} | Abscissa | Ordinate | area | cence | 488 | 488 |
| SA | 240 | 127 | 1228 | 44238 | 1.274 | 56359 |
| IgG | 238 | 195 | 1198 | 54695 | 0.902 | 49335 |
| Diphtheria | 238 | 263 | 1203 | 44069 | 1.691 | 74521 |
| Rubella | 235 | 332 | 1117 | 56298 | 0.075 | 4222 |
| HL | 309 | 123 | 452 | 9652 | 0.458 | 4421 |
| IgM | 306 | 197 | 918 | 18369 | 0.183 | 3362 |
| Tetanus | 304 | 266 | 842 | 26512 | 0.078 | 2068 |
| Measles | 303 | 334 | 1104 | 37075 | 0.13 | 4820 |
| Mumps | 302 | 401 | 1038 | 29978 | 0.118 | 3537 |

TABLE 1B
Serum N^{o} 2

| | | | | | Ratio | Fluores- |
|---|---|---|---|---|---|---|
| | | | Surface | Fluores- | F350/ | cence |
| N^{o} | Abscissa | Ordinate | area | cence | F488 | 488 |
| SA | 249 | 109 | 1199 | 41653 | 1.839 | 76600 |
| IgG | 250 | 177 | 1255 | 49420 | 1.263 | 62417 |
| Diphtheria | 251 | 247 | 1176 | 39874 | 2.350 | 93704 |
| Rubella | 251 | 313 | 1173 | 50395 | 0.108 | 5443 |
| HL | 316 | 106 | 537 | 10853 | 0.627 | 6805 |
| IgM | 318 | 176 | 895 | 15917 | 0.249 | 3963 |
| Tetanus | 321 | 244 | 834 | 21013 | 0.357 | 7502 |
| Measles | 319 | 314 | 1164 | 39486 | 0.118 | 4659 |
| Mumps | 320 | 382 | 1079 | 31342 | 0.120 | 3761 |

TABLE 1C
Serum N^{o} 3

| | | | | | Ratio | Fluores- |
|---|---|---|---|---|---|---|
| | | | Surface | Fluores- | F350/ | cence |
| N^{o} | Abscissa | Ordinate | area | cence | F488 | 488 |
| SA | 210 | 85 | 1175 | 34462 | 1.981 | 68269 |
| IgG | 210 | 153 | 1293 | 47678 | 1.561 | 74425 |
| Diphtheria | 210 | 220 | 1255 | 54523 | 0.342 | 18647 |
| Rubella | 209 | 288 | 1172 | 49101 | 0.073 | 3584 |
| HL | 278 | 78 | 474 | 9180 | 0.740 | 6793 |
| IgM | 278 | 151 | 943 | 15225 | 0.335 | 5100 |
| Tetanus | 278 | 220 | 848 | 24553 | 0.046 | 1129 |
| Measles | 278 | 288 | 1178 | 34879 | 0.153 | 5336 |
| Mumps | 278 | 357 | 1123 | 26949 | 0.095 | 2560 |

TABLE 1D
Serum N^{o} 4

| | | | | | Ratio | Fluores- |
|---|---|---|---|---|---|---|
| | | | Surface | Fluores- | F350/ | cence |
| N^{o} | Abscissa | Ordinate | area | cence | F488 | 488 |
| SA | 240 | 110 | 1202 | 40889 | 1.093 | 44692 |
| IgG | 240 | 177 | 1269 | 53557 | 1.123 | 60145 |
| Diphtheria | 242 | 246 | 1253 | 56284 | 0.316 | 17786 |
| Rubella | 241 | 314 | 1106 | 43970 | 0.107 | 4705 |
| HL | 308 | 106 | 533 | 9932 | 0.797 | 7916 |
| IgM | 308 | 178 | 926 | 17540 | 0.309 | 5420 |
| Tetanus | 309 | 244 | 838 | 29892 | 0.049 | 1465 |
| Measles | 309 | 314 | 1174 | 41689 | 0.093 | 3877 |
| Mumps | 310 | 382 | 1044 | 29666 | 0.110 | 3263 |

1. Serological method for determining the vaccine status of a person by detection and quantification of IgG-type serum antibodies specific to the vaccine antigens of a plurality of pathogenic agents of bacterial, viral, fungal or parasitic type, characterized in that a complex of immunological reactions is detected and quantified between each said vaccine antigen and respectively each said IgG-type antibody specific to said vaccine antigen, possibly present in a sample of human serum to be tested, by conducting the steps of:
1—Contacting one same said serum sample to be tested with:
one same solid substrate on which a plurality of said vaccine antigens are fixed corresponding to a plurality of different pathogenic agents, preferably at least 3, further preferably at least 4 said vaccine antigens of different pathogenic agents, at different areas of the substrate,
in the presence of at least one detection substance reacting by complexing with said IgG-type specific antibodies and not reacting with said vaccine antigens, and
2—The concentration of said IgG-type specific antibodies is determined by quantifying the complexes resulting from the reaction of at least one said detection substance with said IgG-type specific antibodies complexed with said vaccine antigens fixed onto said solid substrate.

2. Method according to claim 1, characterized in that said detection substance contains fluorescent labelling.

3. Method according to claim 2, characterized in that the concentration of said class G immunoglobulins of the patient species specific to said vaccine antigen is determined in the sample to be tested, by automated reading of the intensity of the fluorescent signal emitted by said fluorescent labelling element using appropriate reading apparatus able to quantify the same.

4. Method according to claim 1, characterized in that said vaccine antigens comprise at least 3, preferably 3 to 12 vaccine antigens of different pathogenic agents chosen from among the following viruses: mumps, rubella, measles, chicken pox, poliomyelitis, yellow fever, tick-borne encephalitis, hepatitis A, hepatitis B, and from the following bacteria: Bordetella pertussis, tetanus and diphtheria.

5. Method according to claim 1, characterized in that it is determined whether the concentration of said IgG-type specific antibodies reaches a given threshold on and after which said specific antibody has a protective action protecting against the disease determined by the pathogen.

6. Method according to claim 1, characterized in that one same detection substance is used to detect the different specific antibodies of the different vaccine antigens.

7. Method according to claim 6, characterized in that said detection substance used is an anti-IgG immunoglobulin, preferably a goat or chick immunoglobulin.

8. Method according to claim 1, characterized in that the presence and reactivity of said detection substance is controlled by conducting the steps of:
contacting said sample to be tested with one same said solid substrate, on which a first control antigen has also been fixed which is a class G non-specific immunoglobulin, in the presence of a solution containing a said detection substance, and
verifying whether said detection substance has reacted with said first control antigen fixed on said solid substrate.

9. Method according to claim 1, characterized in that the possible presence of an antinuclear antibody is controlled in said sample to be tested, by:
contacting said sample to be tested with:
one same said solid substrate on which a second control antigen has been fixed which comprises DNA/histone complexes, preferably comprising nuclei of nucleated cells of patient species cells, further preferably all or part of continuous line patient species cells, and
in the presence of a said detection substance consisting of a labelling antibody which only reacts with said class G immunoglobulin of the patient species; and
verifying whether said control antigen fixed on the solid substrate reacts with said detection substance.

10. Method according to claim 1, characterized in that it is controlled that said tested sample effectively contains a serum of the patient species, by detecting whether immunoglobulins of the patient species react with a third control antigen containing protein A of a Staphylococcus aureus bacterium, preferably by contacting said sample with the same said solid substrate on which a said third control antigen is fixed, in the presence of a same said detection substance which is a substance reacting with an immunoglobulin of the patient species and not with said third control antigen, preferably an anti-immunoglobulin antibody of the patient species, and not reacting with protein A.

11. Method according to claim 10, characterized in that said third control antigen is a whole Staphylococcus bacterium containing protein A.

12. Method according to either of claims 10 or 11, characterized in that said detection substance is an anti-IgG goat or chick immunoglobulin, conjugated with a fluorescent substance.

13. Method according to claim 1, characterized in that at least one said vaccine antigen is a corpuscular microbial antigen consisting of a whole inactivated microbe or microbe fraction, preferably fixed onto the solid substrate by simple deposit and physical adsorption or physicochemical binding with the substrate, preferably in a mixture with a protein binder.

14. Method according to claim 1, characterized in that as solid substrate a glass or plastic slide is used, or a flat-bottomed well of a microtitration plate in glass or plastic.

15. Method according to claim 1, characterized in that a determined volume of whole blood is collected using a capillary tube in a bottle containing a determined volume of buffer allowing elution of the serum, the serum then preferably being diluted to a determined concentration, preferably 1/100 to 1/20, and a determined volume of serum so diluted is deposited on the different deposit areas of said control antigens and vaccine antigens on said solid substrate.

16. Method according to claim 1, characterized in that for each detection, and optionally each quantification, of a said vaccine antigen the following measurements are made:
1—a first measurement of a first value representing the quantity of a first labelling element, preferably the first value of the intensity of a signal emitted by said first fluorescent labelling element, said first labelling element fixing non-specifically to any protein in the deposit area of said vaccine antigen, and
2—a second measurement of a second value representing the quantity of a second labelling element, different from said first labelling element and emitting a different signal, preferably a second value of the intensity of the signal emitted by this second fluorescent labelling element at a different excitation wavelength to that of said first fluorescent labelling element, said second labelling element being the labelling element of said detection substance of said vaccine antigen, in the deposit area of said antigen, and
3—the ratio of said first and second values is calculated, and
4—the value of said ratio is compared with value of a reference ratio obtained from a collection of positive and negative reference sera, thereby making it possible to determine, by comparison, whether or not there is a need to vaccinate the person for said vaccine antigen according to the ratio of said first and second values.

17. Kit which can be used to implement the method according to claim 1, characterized in that it contains:
one said same solid substrate on which at least one said plurality of vaccine antigens is fixed, and optionally at least one said control antigen, and
reagents such as a said detection substance and if necessary reagents which can be used to detect said labelling element.

18. Kit according to claim 17, characterized in that it contains:
one said same solid substrate on which at least one said corpuscular vaccine antigen is fixed with said first, second and third control antigens, and
one same said detection substance.

19. Kit according to claim 17 or 18, characterized in that it comprises a bottle containing a determined volume of an elution buffer to collect a determined volume of serum sample to be tested.

20. Method for preparing a solid substrate which can be used in a method or kit according to claim 1, characterized in that on said solid substrate a plurality of said vaccine antigens are deposited and fixed containing at least one corpuscular vaccine antigen in the form of a suspension of whole microbes or microbe fractions—preferably a vaccine antigen in the form of a whole, deactivated, living virus—and optionally said control antigens in the form of a suspension of corpuscles of non-confluent cells or whole bacteria or fractions of cells or bacteria, said corpuscular antigens preferably being deposited by an automated spotter which further preferably comprises a syringe.

21. Method according to claim 20, characterized in that said corpuscular vaccine and/or control antigens are associated with a dye, preferably a fluorescent dye, in the form of a suspension at a concentration to enable their visualization after deposit by means of said dye, allowing verification that said antigens are fixed onto the solid substrate.

22. Method according to claim 21, characterized in that the control antigens in cell suspension form, are calibrated at a concentration of 10⁷ to 10⁹ cells/ml, the suspensions of bacteria or bacteria fractions at a concentration of 10⁷ to 10⁹ particles/ml, and the suspensions of whole viruses at a concentration of 10⁹ to 10¹⁰ particles/ml.

23. Method according to claim 20, characterized in that said control and corpuscular vaccine antigens are deposited in a mixture with a protein binder, stabilizing their fixing to said solid substrate.

24. Method according to claim 23, characterized in that said protein binder is chosen from among egg yolk, gelatine, bovine serum albumin or a non-human polyclonal IgG, preferably goat.

25. Method according to claim 22, characterized in that said corpuscular vaccine antigen is deposited on said solid substrate consisting of a glass slide, in a mixture with a goat polyclonal IgG-type immunoglobulin.

26. Method according to claim 20, characterized in that prior washing of said solid substrate is performed with a solution of an ethanol/acetone mixture, preferably 50-50, then said antigens are deposited and their fixing stabilized by physical adsorption on said solid substrate by treating with alcohol, preferably methanol or ethanol, which alcohol is subsequently removed, and further preferably the fixing of said antigens is verified by staining, preferably by fluorescent labelling that is non-specific to the proteins or DNA.
